(19) United States
(12) Patent Application Publication
(10) Pub. No.: US 2022/0074943 A1
(43) Pub. Date: Mar. 10, 2022

(54) COMPOSITION FOR DIAGNOSING BONE METASTASIS OF CANCER AND METHOD FOR DIAGNOSING BONE METASTASIS OF CANCER USING SAME

(71) Applicant: KOREA UNIVERSITY RESEARCH AND BUSINESS FOUNDATION, Seoul (KR)

(72) Inventors: Serkin PARK, Seoul (KR); Kyoung Jin LEE, Seoul (KR)

(21) Appl. No.: 17/419,481

(22) PCT Filed: Dec. 23, 2019
(86) PCT No.: PCT/KR2019/018328

§ 371 Date: Jun. 29, 2021

(30) Foreign Application Priority Data
Jan. 3, 2019 (KR) 10-2019-0000891

(51) Int. Cl. G01N 33/574

(52) U.S. Cl. CPC G01N 33/57484; G01N 2333/70553; G01N 2500/00; G01N 2333/70596; G01N 2333/70589

(57) ABSTRACT

The present invention relates to a composition for diagnosing bone metastasis of cancer, a method for providing information needed for diagnosis of bone metastasis of cancer using same, a method for providing information needed for monitoring responses to treatment of bone metastasis of cancer using same, and a method for screening a therapeutic agent for bone metastasis of cancer using same. The composition for diagnosing bone metastasis of cancer of the present invention has the effect of effectively diagnosing bone metastasis of cancer at an early stage.

### TECHNICAL FIELD

The present disclosure was made with the support of the Ministry of Health and Welfare, Republic of Korea, under Project No. HA17C0040, which was conducted by the Seoul National University Hospital in the research project named “Development of Cell-based biomarkers for predicting bone metastasis” under management of the National Cancer Center of Korea for project name “the National R&D Program for Cancer Control”, from Jan. 1, 2018 to 31 Dec. 2018.

This application claims priority to and the benefit of Korean Patent Application No. 10-2019-0000891 filed in the Korean Intellectual Property Office on 3 Jan. 2019, the contents of which are incorporated herein in its entirety by reference.

The present disclosure relates to a composition for diagnosis of cancer bone metastasis, a method for providing information necessary for diagnosis of cancer bone metastasis using same, a method for providing information for monitoring a response to therapy for cancer bone metastasis using same, and a method for screening a therapeutic agent for cancer bone metastasis using same. The composition for diagnosis of cancer bone metastasis of the present disclosure can effectively diagnose cancer bone metastasis in an early stage.

### BACKGROUND ART

Cancer metastasis is difficult to treat and has emerged as an important clinical problem that leads directly to poor quality of life and death for patients. Organs for which metastasis commonly occurs include bones, lungs, and the liver. Among others, bone metastasis occurs when tumor cells relocate to the bone, and is a common terminal symptom of breast cancer, prostate cancer, lung cancer, kidney cancer, thyroid cancer, etc. Bone metastasis, once diagnosed, causes rapid poor prognosis and makes it difficult to treat the cancer by surgery. Also, there is no way to prevent bone metastasis from progressing. A major reason for this poor treatment or prognosis for bone metastasis is that bone metastasis can be found only after extensive progression as the current diagnosis of bone diagnosis relies on radiologic examinations or a small number of blood osteoclast markers. For breast cancer, for example, hormone suppression therapy and periodic bone scans (radiologic examination on bone metastasis using radiation markers) are performed at a five-year follow-up time following initial diagnosis and surgery, chemotherapy, etc. Even when there are no symptoms of pain, fracture, or nerve compression, diagnosis through bone scanning revealed already significantly progressed bone destruction in most cases, finally resulting in poor prognosis.

Bone metastasis proceeds as follows. Cancer cells come off their primary tumor, circulate along the bloodstream, and reach a bone. This process is called seeding. Seeded cancer cells soon enter the resting phase and remain at rest for years or decades. Thereafter, for unknown reasons, the resting cancer cells become active and undergo cell division to proceed to the clinical bone metastasis stage. In the resting phase, cancer cells exist only in the bone marrow, with no interactions between the cancer cells and the bone marrow cells. However, in the bone micrometastasis stage, cancer cells begin to interact with nearby bone marrow cells and bone cells, and proliferate actively. In the clinical bone metastasis stage, the bone tissue is destroyed by cancer cells, osteoclasts, and the like, with consequent development of symptoms such as pain and fracture.

Conventional bone metastasis diagnosis using radiologic examinations or bone markers are possible only in the clinical bone metastasis stage. In this case, the tumor has significantly advanced, with difficulty in effective therapy therefor. This is because the diagnosis is possible only after destruction of the bone tissue arises. Furthermore, conventional examination methods are unable to measure therapeutic responses to therapeutic agents for bone metastasis and have a difficulty in screening efficacies of newly developed therapeutic agents for bone metastasis. Therefore, there is an increasing need for a diagnostic composition or kit that can advance an initiation time of therapy for bone metastasis through early diagnosis prior to destruction of bone tissues in the bone micrometastasis stage, can monitoring whether a therapeutic agent for bone metastasis is efficacious, and can screen therapeutic agents for bone metastasis, and a method for providing information for the diagnosis.

### SUMMARY

### Technical Problem

An aspect of the present disclosure is to provide a composition for diagnosis of cancer bone metastasis.

Another aspect of the present disclosure is to provide a method for providing information necessary for diagnosis of cancer bone metastasis.

A further aspect of the present disclosure is to provide a method for providing information for monitoring a response to therapy for cancer bone metastasis.

A still further aspect of the present disclosure is to provide a method for screening a therapeutic agent for cancer bone metastasis.

### Solution to Problem

The present disclosure relates to a composition for diagnosis of cancer bone metastasis, a method for providing information necessary for diagnosis of cancer bone metastasis using same, a method for providing information for monitoring a response to therapy for cancer bone metastasis using same, and a method for screening a therapeutic agent for cancer bone metastasis using same. The present disclosure can more simply diagnose bone metastasis, through a blood test, but not through radiologic diagnosis methods including bone scanning, enables early diagnosis compared to radiologic diagnosis methods, and allows monitoring therapy prognosis for cancer bone metastasis, thereby greatly improving survival rates and quality of life in cancer bone metastasis patients.

Below, a detailed description will be given of the present disclosure.

An aspect of the present disclosure pertains to a composition for diagnosis of cancer bone metastasis.

In the present disclosure, the composition may comprise the following detecting agents:

a CD45 detecting agent, a HLA-DR detecting agent, and a CD11 b detecting agent; and

(a) a CD14 detecting agent and a CCR2 detecting agent, (b) a CD14 detecting agent, (c) a CD15 detecting agent and a CD33 detecting agent, or (d) a CD14 detecting agent, a CCR2 detecting agent, a CD15 detecting agent, and a CD33 detecting agent.

In an embodiment of the present disclosure, the composition may comprise a CD45 detecting agent, a HLA-DR detecting agent, a CD11 b detecting agent, a CD14 detecting agent, and a CCR2 detecting agent.

In another embodiment of the present disclosure, the composition may comprise a CD45 detecting agent, a HLA-DR detecting agent, a CD11b detecting agent, a CD14 detecting agent, a CD15 detecting agent, and a CD33 detecting agent.

In another embodiment of the present disclosure, the composition may comprise a CD45 detecting agent, a HLA-DR detecting agent, a CD11b detecting agent, a CD14 detecting agent, a CCR2 detecting agent, a CD15 detecting agent, and a CD33-positive detecting agent.

In an embodiment of the present disclosure, the detecting agents may each be selected from the group consisting of an antibody, an aptamer, DNA, RNA, a protein, and a polypeptide, and for example may be an antibody, but with no limitations thereto.

As used herein, the term “antibody”, also known as an immunoglobulin immunologically responsive to a specific antigen, refers to a protein molecule specifically recognizing and serving as a receptor for an antigen and is intended to encompass polyclonal antibodies, monoclonal antibodies, whole antibodies, and antibody fragments.

As used herein, “whole antibody” refers to an intact immunoglobulin having a structure composed of two full-length light chains and two full-length heavy chains, with linkages between the light changes and the heavy chains via disulfide bonds.

Whole antibodies are divided into classes IgA, IgD, IgE, IgM, and IgG, IgG being further subdivided into subtypes IgG1, IgG2, IgG3, and IgG4.

As used herein, the term “antibody fragment” refers to a part of a whole antibody, which retains an antigen-binding function. Examples include, but are not limited to, an Fc fragment, Fab, Fab′, F(ab′)₂, and Fv.

“Fc fragment” refers to an end region of an antibody, which is able to bind to a cell surface receptor, such as an Fc receptor and is composed of the second or third constant domains of two heavy chains.

Fab has a structure possessing light chain and heavy chain variable regions, a light chain constant region, and a heavy chain first constant domain (CH1), and has one antigen-binding site.

Fab′ is different from Fab in that the former has a hinge region including one or more cysteine residues at the C-terminus of the heavy chain CH1 domain.

An F(ab′)₂ antibody is generated through a disulfide bond formed between the cysteine residues in the hinge region of Fab′.

Fv (variable fragment) refers to a minimal antibody fragment having only a heavy chain variable region and a light chain variable region.

The disulfide-stabilized variable fragment (dsFv) has a structure in which a heavy chain variable region and a light chain variable region are linked to each other by a disulfide bond, while the single chain variable fragment (scFV) generally has a structure in which a heavy chain variable region (VH) and a light chain variable region (VL) are covalently linked to each other by a peptide linker.

In an embodiment of the present disclosure, the detecting agents may each be independently labeled.

In an embodiment of the present disclosure, the detecting agents may be labeled with at least one marker selected from the group consisting of a ligand, a bead, a radionuclide, an enzyme, a substrate, a cofactor, an inhibitor, a fluorescer, a fluorescent protein, a chemiluminescent substance, a magnetic particle, a hapten, and a dye, but without limitations thereto. So long as it is known in the art, any detectable marker may be used. A person skilled in the art could select markers suitable for purposes of the present disclosures.

Examples of the ligand include, but are not limited to, biotin, avidin, and streptoavidin.

The enzyme may be exemplified by luciferase, peoxidase, and beta galactosidase, but with no limitations thereto.

The fluorescer may be fluorescein, coumarin, rhodamine, phycoerythrin, and sulforhodamine acid, sulforhodamine acid chloride (Texas red), etc., but is not limited thereto.

The fluorescent protein may be at least one selected from the group consisting of red fluorescent protein (RFP), enhanced red fluorescent protein (ERFP), green fluorescent protein (GFP), modified green fluorescent protein (GFP), enhanced GFP, blue fluorescent protein (BFP), enhanced blue fluorescent protein (eBFP), cyan fluorescent protein (CFP), enhanced cyan fluorescent protein (eCFP), yellow fluorescent protein (YFP), and enhanced yellow fluorescent protein (eYFP), but is not limited thereto.

In an embodiment of the present disclosure, the detecting agents may each be a specific antibody to a subject to be detected.

As used herein, the term “specific antibody to a subject to be detected” refers to an antibody or a fragment thereof that recognizes as an antigen a subject to be detected and binds thereto.

In an embodiment of the present disclosure, the CD45 detecting agent may be an anti-CD45 antibody, for example, CD45 PerPC-Cy5.5 (BD #564105), but is not limited thereto.

In an embodiment of the present disclosure, the HLA-DR detecting agent may be an anti-HLA-DR, for example, HLA-DR Alexa Fluor® 700 (BD #560743), but is not limited thereto.

In an embodiment of the present disclosure, the CD11 b detecting agent may be an anti-CD11 b antibody, for example, CD11 b APC (Allophycocyanin) (BD #55019), but is not limited thereto.

In an embodiment of the present disclosure, the CD14 detecting agent may be an anti-CD14 antibody, for example, CD14 APC-Cyanine 7 (APC-Cy 7) (BD #557831), but is not limited thereto.

In an embodiment of the present disclosure, the CD15 detecting agent may be an anti-CD15 antibody, for example, CD15 FITC (Fluorescein isothiocyanate) (BD #555401), but is not limited thereto.

In an embodiment of the present disclosure, the CD33 detecting agent may be an anti-CD33 antibody, for example, CD33 PE-Cy7 (Phycoerythrin-Cyanine 7) (BD #333946), but is not limited thereto.

In an embodiment of the present disclosure, the CCR2 detecting agent may be an anti-CCR2 antibody, for example, CCR2 PE (Phycoerythrin) (Biolegend #357206), but is not limited thereto.

In an embodiment of the present disclosure, the detecting agents contained in the composition may each have a concentration of 5 μl/2×10⁵ cells or more, for example, 5 μl/2×10⁵ cells. This concentration is required as a minimum quantity necessary for acquiring a significant data as analyzed by flow cytometry.

In an embodiment of the present disclosure, the cancer may be breast cancer, prostate cancer, liver cancer, lung cancer, bladder cancer, stomach cancer, uterine cancer, colorectal cancer, colon cancer, blood cancer, ovarian cancer, pancreatic cancer, spleen cancer, testis cancer, thymic carcinoma, brain cancer, esophageal cancer, kidney cancer, cholangiocarcinoma, ovarian cancer, thyroid cancer, or skin cancer, for example, breast cancer or prostate cancer, but is not limited thereto.

Another aspect of the present disclosure pertains to a method for providing information necessary for diagnosis of cancer bone metastasis, the method comprising:

a labeling step of contacting a composition for diagnosis of cancer bone metastasis with a sample;

an acquiring step of acquiring a labeled cell; and

an analyzing step of analyzing the labeled cell.

The composition for diagnosis of cancer bone metastasis is as defined above.

In an embodiment of the present disclosure, the sample may be isolated from a patient.

In an embodiment of the present disclosure, the patient may be a mammal, for example, a primate such as a human or a monkey, or a rodent such as a rat, and particularly, a human.

In an embodiment of the present disclosure, the sample may be blood, for example, peripheral blood, but is not limited thereto.

In an embodiment of the present disclosure, the sample may include a cell.

In an embodiment of the present disclosure, the cell may be a mononuclear cell, for example, a myeloid-derived suppressor cell, but is not limited thereto.

In an embodiment of the present disclosure, the cell may be a peripheral blood mononuclear cell obtained from a peripheral blood sample.

In an embodiment of the present disclosure, the sample may be obtained through the following steps:

a first centrifugation step for centrifuging a blood sample taken from a patient;

a mononuclear cell layer acquiring step;

a mononuclear cell layer washing step; and

a second centrifugation step.

The first centrifugation step may be conducted at a temperature of 4 to 25° C., 4 to 22° C., 4 to 19° C., 4 to 16° C., 4 to 13° C., 4 to 10° C., 4 to 7° C., or 4 to 5° C., for example, 4° C., but with no limitations thereto.

In addition, the first centrifugation step may be conducted at 500 to 2000×g, 500 to 1800×g, 500 to 1600×g, 500 to 1400×g, 500 to 1200×g, 500 to 1000×g, 500 to 800×g, 600 to 2000×g, 600 to 1800×g, 600 to 1600×g, 600 to 1400×g, 600 to 1200×g, 600 to 1000×g, or 600 to 800×g, for example, 635×g, but with no limitations thereto.

Also, the first centrifugation may be conducted for 5 to 60 minutes, 5 to 55 minutes, 5 to 50 minutes, 5 to 45 minutes, 5 to 40 minutes, 5 to 35 minutes, 5 to 30 minutes, 5 to 25 minutes, 10 to 60 minutes, 10 to 55 minutes, 10 to 50 minutes, 10 to 45 minutes, 10 to 40 minutes, 10 to 35 minutes, 10 to 30 minutes, 10 to 25 minutes, 15 to 60 minutes, 15 to 55 minutes, 15 to 50 minutes, 15 to 45 minutes, 15 to 40 minutes, 15 to 35 minutes, 15 to 30 minutes, 15 to 25 minutes, for example, 20 minutes, but with no limitations thereto.

In an embodiment of the present disclosure, the first centrifugation may be conducted at 4° C. and 635×g for 20 minutes.

The washing step may be conducted with at least one selected from the group consisting of phosphate buffered saline (PBS), physiological saline, and a FACS wash buffer (buffer containing fetal bovine serum or bovine serum albumin and ethylenediaminetetraacetic acid (EDTA)), for example, a FACS wash buffer, with no limitations thereto.

The second centrifugation may be conducted at 4 to 25° C., 4 to 22° C., 4 to 19° C., 4 to 16° C., 4 to 13° C., 4 to 10° C., 4 to 7° C., or 4 to 5° C., for example, at 4° C., but with no limitations thereto.

In addition, the second centrifugation may be conducted at 500 to 2000×g, 500 to 1800×g, 500 to 1600×g, 500 to 1400×g, 500 to 1200×g, 500 to 1000×g, 500 to 800×g, 600 to 2000×g, 600 to 1800×g, 600 to 1600×g, 600 to 1400×g, 600 to 1200×g, 600 to 1000×g, or 600 to 800×g, for example, 783×g, with no limitations thereto.

In addition, the second centrifugation may be conducted for 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 3 to 60 minutes, 3 to 55 minutes, 3 to 50 minutes, 3 to 45 minutes, 3 to 40 minutes, 3 to 35 minutes, 3 to 30 minutes, 3 to 25 minutes, 3 to 20 minutes, 3 to 15 minutes, or 3 to 10 minutes, for example, 5 minutes, but with no limitations thereto.

In a concrete embodiment of the present disclosure, the second centrifugation may be conducted at 4° C. and 783×g for 5 minutes.

In an embodiment of the present disclosure, the labeling step may be conducted for 5 to 60 minutes, 5 to 55 minutes, 5 to 50 minutes, 5 to 45 minutes, 5 to 40 minutes, 5 to 35 minutes, 5 to 30 minutes, 5 to 25 minutes, 10 to 60 minutes, 10 to 55 minutes, 10 to 50 minutes, 10 to 45 minutes, 10 to 40 minutes, 10 to 35 minutes, 10 to 30 minutes, 10 to 25 minutes, 15 to 60 minutes, 15 to 55 minutes, 15 to 50 minutes, 15 to 45 minutes, 15 to 40 minutes, 15 to 35 minutes, 15 to 30 minutes, or 15 to 25 minutes, for example, 20 minutes, but with no limitations thereto.

In an embodiment of the present invention, the labeling step may be conducted at 4 to 25° C., 4 to 22° C., 4 to 19° C., 4 to 16° C., 4 to 13° C., 4 to 10° C., 4 to 7° C., or 4 to 5° C., for example, 4° C., with no limitations thereto.

In an embodiment of the present disclosure, the sample may contain 2×10⁵ or more mononuclear cells. This concentration is required as a minimum quantity necessary for acquiring a significant data as analyzed by flow cytometry.

In an embodiment of the present disclosure, the labeled mononuclear cells acquiring step may include the following steps:

a step of washing with a buffer; and

a third centrifugation step.

The buffer may be at least one selected from the group consisting of phosphate buffered saline (PBS), physiological saline, and a FACS wash buffer (buffer containing fetal bovine serum or bovine serum albumin and ethylenediaminetetraacetic acid (EDTA)), but is not limited thereto.

The third centrifugation may be conducted at 4 to 25° C., 4 to 22° C., 4 to 19° C., 4 to 16° C., 4 to 13° C., 4 to 10° C., 4 to 7° C., or 4 to 5° C., for example, at 4° C., but with no limitations thereto.

The third centrifugation may be conducted at 400 to 2000×g, 400 to 1800×g, 400 to 1600×g, 400 to 1400×g, 400 to 1200×g, 400 to 1000×g, or 400 to 800×g, for example, at 441×g, but with no limitations thereto.

In addition, the third centrifugation may be conducted for 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 3 to 60 minutes, 3 to 55 minutes, 3 to 50 minutes, 3 to 45 minutes, 3 to 40 minutes, 3 to 35 minutes, 3 to 30 minutes, 3 to 25 minutes, 3 to 20 minutes, 3 to 15 minutes, or 3 to 10 minutes, for example, 5 minutes, but with no limitations thereto.

In an embodiment of the present disclosure, the third centrifugation may be conducted at 4° C. and 441×g for 5 minutes.

In an embodiment of the present disclosure, the labeled mononuclear cell analyzing step may include the following steps of:

obtaining only single cells;

sorting the cells to give a CD45-positive cell population;

reclassifying only the CD45-positive cell population to give a HLA-DR-negative cell population;

reclassifying only the HLA-DR-negative cell population to give a CD11b-positive cell population; and

reclassifying only the CD11b-positive cell population to give a CD14-positive and CCR2-positive cell population.

As used herein, the term “single cells” refers to cells that are not in a cluster state, but in an individually separate condition.

The analysis may be performed through immunochromatography, immunohistochemical staining, enzyme-linked immunosorbent assay (ELISA), radioimmunoassay (RIA), enzyme immunoassay (EIA), florescence immunoassay (FIA), luminescence immunoassay (LIA), Western blotting, fluorescence activated cell sorter (FACS), or flow cytometry, for example, flow cytometry, but with no limitations thereto.

As used herein, the term “flow cytometry” refers to a technique used to count cells, sort cells, or probe biomarkers. By the technique, cells labeled with a fluorescent or an isotope can be detected.

As used herein, “immunoassay” refers to a biochemical test method for measuring the presence or concentration of a polymer in a solution with the aid of an antibody or immunoglobulin, and includes, for example, radioimmunoassay (RIA), enzyme-linked immunosorbent assay (ELISA), immunofluorescence, and chemiluminescent-linked immunoassay.

The assay method may be used for quantitative analysis of cells by counting cells conjugated with the detecting agent or by measuring concentrations of the cells.

In an embodiment of the present disclosure, the cancer may be breast cancer, prostate cancer, liver cancer, lung cancer, bladder cancer, stomach cancer, uterine cancer, colorectal cancer, colon cancer, blood cancer, ovarian cancer, pancreatic cancer, spleen cancer, testis cancer, thymic carcinoma, brain cancer, esophageal cancer, kidney cancer, cholangiocarcinoma, ovarian cancer, thyroid cancer, or skin cancer, for example, breast cancer or prostate cancer, but is not limited thereto.

Another aspect of the present disclosure pertains to a method for providing information for monitoring a response to therapy for cancer bone metastasis, the method comprising:

a labeling step of contacting a composition for diagnosis of cancer bone metastasis with a sample;

an acquiring step of acquiring a labeled mononuclear cell; and

an analyzing step of analyzing the labeled mononuclear cell.

The composition for diagnosis of cancer bone metastasis is as defined above.

In an embodiment of the present disclosure, the sample may be isolated from a patient, for example, a patient that is being treated.

In an embodiment of the present disclosure, the patient may be a mammal, for example, a primate such as a human or a monkey, or a rodent such as a rat, and particularly, a human.

In an embodiment of the present disclosure, the sample may be blood, for example, peripheral blood, but is not limited thereto.

In an embodiment of the present disclosure, the sample may include a cell.

In an embodiment of the present disclosure, the cell may be a mononuclear cell, for example, a myeloid-derived suppressor cell, but is not limited thereto.

In an embodiment of the present disclosure, the cell may be a peripheral blood mononuclear cell obtained from a peripheral blood sample.

In an embodiment of the present disclosure, the sample may be obtained through the following steps:

a first centrifugation step for centrifuging a blood sample taken from a patient;

a mononuclear cell layer acquiring step;

a mononuclear cell layer washing step; and

a second centrifugation step.

The first centrifugation step may be conducted at a temperature of 4 to 25° C., 4 to 22° C., 4 to 19° C., 4 to 16° C., 4 to 13° C., 4 to 10° C., 4 to 7° C., or 4 to 5° C., for example, 4° C., but with no limitations thereto.

In addition, the first centrifugation step may be conducted at 500 to 2000×g, 500 to 1800×g, 500 to 1600×g, 500 to 1400×g, 500 to 1200×g, 500 to 1000×g, 500 to 800×g, 600 to 2000×g, 600 to 1800×g, 600 to 1600×g, 600 to 1400×g, 600 to 1200×g, 600 to 1000×g, or 600 to 800×g, for example, 635×g, but with no limitations thereto.

Also, the first centrifugation may be conducted for 5 to 60 minutes, 5 to 55 minutes, 5 to 50 minutes, 5 to 45 minutes, 5 to 40 minutes, 5 to 35 minutes, 5 to 30 minutes, 5 to 25 minutes, 10 to 60 minutes, 10 to 55 minutes, 10 to 50 minutes, 10 to 45 minutes, 10 to 40 minutes, 10 to 35 minutes, 10 to 30 minutes, 10 to 25 minutes, 15 to 60 minutes, 15 to 55 minutes, 15 to 50 minutes, 15 to 45 minutes, 15 to 40 minutes, 15 to 35 minutes, 15 to 30 minutes, 15 to 25 minutes, for example, 20 minutes, but with no limitations thereto.

In an embodiment of the present disclosure, the first centrifugation may be conducted at 4° C. and 635×g for 20 minutes.

The washing step may be conducted with at least one selected from the group consisting of phosphate buffered saline (PBS), physiological saline, and a FACS wash buffer (buffer containing fetal bovine serum or bovine serum albumin and ethylenediaminetetraacetic acid (EDTA)), for example, a FACS wash buffer, with no limitations thereto.

The second centrifugation may be conducted at 4 to 25° C., 4 to 22° C., 4 to 19° C., 4 to 16° C., 4 to 13° C., 4 to 10° C., 4 to 7° C., or 4 to 5° C., for example, at 4° C., but with no limitations thereto.

In addition, the second centrifugation may be conducted at 500 to 2000×g, 500 to 1800×g, 500 to 1600×g, 500 to 1400×g, 500 to 1200×g, 500 to 1000×g, 500 to 800×g, 600 to 2000×g, 600 to 1800×g, 600 to 1600×g, 600 to 1400×g, 600 to 1200×g, 600 to 1000×g, or 600 to 800×g, for example, 783×g, with no limitations thereto.

In addition, the second centrifugation may be conducted for 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 3 to 60 minutes, 3 to 55 minutes, 3 to 50 minutes, 3 to 45 minutes, 3 to 40 minutes, 3 to 35 minutes, 3 to 30 minutes, 3 to 25 minutes, 3 to 20 minutes, 3 to 15 minutes, or 3 to 10 minutes, for example, 5 minutes, but with no limitations thereto.

In a concrete embodiment of the present disclosure, the second centrifugation may be conducted at 4° C. and 783×g for 5 minutes.

In an embodiment of the present disclosure, the labeling step may be conducted for 5 to 60 minutes, 5 to 55 minutes, 5 to 50 minutes, 5 to 45 minutes, 5 to 40 minutes, 5 to 35 minutes, 5 to 30 minutes, 5 to 25 minutes, 10 to 60 minutes, 10 to 55 minutes, 10 to 50 minutes, 10 to 45 minutes, 10 to 40 minutes, 10 to 35 minutes, 10 to 30 minutes, 10 to 25 minutes, 15 to 60 minutes, 15 to 55 minutes, 15 to 50 minutes, 15 to 45 minutes, 15 to 40 minutes, 15 to 35 minutes, 15 to 30 minutes, or 15 to 25 minutes, for example, 20 minutes, but with no limitations thereto.

In an embodiment of the present invention, the labeling step may be conducted at 4 to 25° C., 4 to 22° C., 4 to 19° C., 4 to 16° C., 4 to 13° C., 4 to 10° C., 4 to 7° C., or 4 to 5° C., for example, 4° C., with no limitations thereto.

In an embodiment of the present disclosure, the sample may contain 2×10⁵ or more mononuclear cells. This concentration is required as a minimum quantity necessary for acquiring a significant data as analyzed by flow cytometry.

In an embodiment of the present disclosure, the labeled mononuclear cells acquiring step may include the following steps:

a step of washing with a buffer; and

a third centrifugation step.

The buffer may be at least one selected from the group consisting of phosphate buffered saline (PBS), physiological saline, and a FACS wash buffer (buffer containing fetal bovine serum or bovine serum albumin and ethylenediaminetetraacetic acid (EDTA)), but is not limited thereto.

The third centrifugation may be conducted at 4 to 25° C., 4 to 22° C., 4 to 19° C., 4 to 16° C., 4 to 13° C., 4 to 10° C., 4 to 7° C., or 4 to 5° C., for example, at 4° C., but with no limitations thereto.

The third centrifugation may be conducted at 400 to 2000×g, 400 to 1800×g, 400 to 1600×g, 400 to 1400×g, 400 to 1200×g, 400 to 1000×g, or 400 to 800×g, for example, at 441×g, but with no limitations thereto.

In addition, the third centrifugation may be conducted for 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 3 to 60 minutes, 3 to 55 minutes, 3 to 50 minutes, 3 to 45 minutes, 3 to 40 minutes, 3 to 35 minutes, 3 to 30 minutes, 3 to 25 minutes, 3 to 20 minutes, 3 to 15 minutes, or 3 to 10 minutes, for example, 5 minutes, but with no limitations thereto.

In an embodiment of the present disclosure, the third centrifugation may be conducted at 4° C. and 441×g for 5 minutes.

In an embodiment of the present disclosure, the labeled mononuclear cell analyzing step may include the following steps of:

obtaining only single cells;

sorting the cells to give a CD45-positive cell population;

reclassifying the cells to give a HLA-DR-negative cell population;

reclassifying the cells to give a CD11b-positive cell population; and

reclassifying the cells to give a CD14-positive and CCR2-positive cell population.

The analysis may be performed through immunochromatography, immunohistochemical staining, enzyme-linked immunosorbent assay (ELISA), radioimmunoassay (RIA), enzyme immunoassay (EIA), florescence immunoassay (FIA), luminescence immunoassay (LIA), Western blotting, fluorescence activated cell sorter (FACS), or flow cytometry, for example, flow cytometry, but with no limitations thereto.

As used herein, the term “flow cytometry” refers to a technique used to count cells, sort cells, or probe biomarkers. By the technique, cells labeled with a fluorescent or an isotope can be detected.

As used herein, “immunoassay” refers to a biochemical test method for measuring the presence or concentration of a polymer in a solution with the aid of an antibody or immunoglobulin, and includes, for example, radioimmunoassay (RIA), enzyme-linked immunosorbent assay (ELISA), immunofluorescence, and chemiluminescent-linked immunoassay.

The assay method may be used for quantitative analysis of cells by counting cells conjugated with the detecting agent or by measuring concentrations of the cells.

In an embodiment of the present disclosure, the cancer may be breast cancer, prostate cancer, liver cancer, lung cancer, bladder cancer, stomach cancer, uterine cancer, colorectal cancer, colon cancer, blood cancer, ovarian cancer, pancreatic cancer, spleen cancer, testis cancer, thymic carcinoma, brain cancer, esophageal cancer, kidney cancer, cholangiocarcinoma, ovarian cancer, thyroid cancer, or skin cancer, for example, breast cancer or prostate cancer, but is not limited thereto.

Another aspect of the present disclosure pertains to a method for screening a therapeutic agent for cancer bone metastasis, the method comprising:

a step of contacting a test substance with a sample;

a labeling step of contacting a composition for diagnosis of cancer bone metastasis with the sample;

an acquiring step of acquiring a labeled mononuclear cell; and

an analyzing step of analyzing the labeled mononuclear cell.

The test substance may be at least one selected from the group consisting of a natural compound, a synthetic compound, RNA, DNA, a polypeptide, an enzyme, a protein, a ligand, an antibody, an antigen, a bacterial or fungal metabolite, and a bioactive molecule, but is not limited thereto.

The test substance may be acquired from libraries of synthetic or natural compounds. Such compound libraries may be constructed using methods known in the art.

By way of example, synthetic compound libraries can be commercially purchased from Maybridge Chemical Co. (UK), Comgenex (USA), Brandon Associates (USA), Microsource (USA), and Sigma-Aldrich (USA), and natural compound libraries can be commercially available from Pan Laboratories (USA) and MycoSearch (USA).

The composition for diagnosis of cancer bone metastasis is as described above.

In an embodiment of the present disclosure, the sample may be a cell found to be cancerous, an extract therefrom, or a culture thereof, but with no limitations thereto.

In an embodiment of the present disclosure, the sample may be isolated from a patient.

In an embodiment of the present disclosure, the patient may be a mammal, for example, a primate such as a human or a monkey, or a rodent such as a rat, and particularly, a human.

In an embodiment of the present disclosure, the sample may be blood, for example, peripheral blood, but is not limited thereto.

In an embodiment of the present disclosure, the sample may include a cell.

In an embodiment of the present disclosure, the cell may be a mononuclear cell, for example, a myeloid-derived suppressor cell, but is not limited thereto.

In an embodiment of the present disclosure, the cell may be a peripheral blood mononuclear cell.

In an embodiment of the present disclosure, the sample may be obtained through the following steps:

a first centrifugation step for centrifuging a blood sample taken from a patient, a cell found to be cancerous, an extract therefrom, or a culture thereof;

a mononuclear cell layer acquiring step;

a mononuclear cell layer washing step; and

a second centrifugation step.

The first centrifugation step may be conducted at a temperature of 4 to 25° C., 4 to 22° C., 4 to 19° C., 4 to 16° C., 4 to 13° C., 4 to 10° C., 4 to 7° C., or 4 to 5° C., for example, 4° C., but with no limitations thereto.

In addition, the first centrifugation step may be conducted at 500 to 2000×g, 500 to 1800×g, 500 to 1600×g, 500 to 1400×g, 500 to 1200×g, 500 to 1000×g, 500 to 800×g, 600 to 2000×g, 600 to 1800×g, 600 to 1600×g, 600 to 1400×g, 600 to 1200×g, 600 to 1000×g, or 600 to 800×g, for example, 635×g, but with no limitations thereto.

Also, the first centrifugation may be conducted for 5 to 60 minutes, 5 to 55 minutes, 5 to 50 minutes, 5 to 45 minutes, 5 to 40 minutes, 5 to 35 minutes, 5 to 30 minutes, 5 to 25 minutes, 10 to 60 minutes, 10 to 55 minutes, 10 to 50 minutes, 10 to 45 minutes, 10 to 40 minutes, 10 to 35 minutes, 10 to 30 minutes, 10 to 25 minutes, 15 to 60 minutes, 15 to 55 minutes, 15 to 50 minutes, 15 to 45 minutes, 15 to 40 minutes, 15 to 35 minutes, 15 to 30 minutes, 15 to 25 minutes, for example, 20 minutes, but with no limitations thereto.

In an embodiment of the present disclosure, the first centrifugation may be conducted at 4° C. and 635×g for 20 minutes.

The washing step may be conducted with at least one selected from the group consisting of phosphate buffered saline (PBS), physiological saline, and a FACS wash buffer (buffer containing fetal bovine serum or bovine serum albumin and ethylenediaminetetraacetic acid (EDTA)), for example, a FACS wash buffer, with no limitations thereto.

The second centrifugation may be conducted at 4 to 25° C., 4 to 22° C., 4 to 19° C., 4 to 16° C., 4 to 13° C., 4 to 10° C., 4 to 7° C., or 4 to 5° C., for example, at 4° C., but with no limitations thereto.

In addition, the second centrifugation may be conducted at 500 to 2000×g, 500 to 1800×g, 500 to 1600×g, 500 to 1400×g, 500 to 1200×g, 500 to 1000×g, 500 to 800×g, 600 to 2000×g, 600 to 1800×g, 600 to 1600×g, 600 to 1400×g, 600 to 1200×g, 600 to 1000×g, or 600 to 800×g, for example, 783×g, with no limitations thereto.

In addition, the second centrifugation may be conducted for 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 3 to 60 minutes, 3 to 55 minutes, 3 to 50 minutes, 3 to 45 minutes, 3 to 40 minutes, 3 to 35 minutes, 3 to 30 minutes, 3 to 25 minutes, 3 to 20 minutes, 3 to 15 minutes, or 3 to 10 minutes, for example, 5 minutes, but with no limitations thereto.

In a concrete embodiment of the present disclosure, the second centrifugation may be conducted at 4° C. and 783×g for 5 minutes.

In an embodiment of the present disclosure, the labeling step may be conducted for 5 to 60 minutes, 5 to 55 minutes, 5 to 50 minutes, 5 to 45 minutes, 5 to 40 minutes, 5 to 35 minutes, 5 to 30 minutes, 5 to 25 minutes, 10 to 60 minutes, 10 to 55 minutes, 10 to 50 minutes, 10 to 45 minutes, 10 to 40 minutes, 10 to 35 minutes, 10 to 30 minutes, 10 to 25 minutes, 15 to 60 minutes, 15 to 55 minutes, 15 to 50 minutes, 15 to 45 minutes, 15 to 40 minutes, 15 to 35 minutes, 15 to 30 minutes, or 15 to 25 minutes, for example, 20 minutes, but with no limitations thereto.

In an embodiment of the present invention, the labeling step may be conducted at 4 to 25° C., 4 to 22° C., 4 to 19° C., 4 to 16° C., 4 to 13° C., 4 to 10° C., 4 to 7° C., or 4 to 5° C., for example, 4° C., with no limitations thereto.

In an embodiment of the present disclosure, the sample may contain 2×10⁵ or more mononuclear cells. This concentration is required as a minimum quantity necessary for acquiring a significant data as analyzed by flow cytometry.

In an embodiment of the present disclosure, the labeled mononuclear cells acquiring step may include the following steps:

a step of washing with a buffer; and

a third centrifugation step.

The buffer may be at least one selected from the group consisting of phosphate buffered saline (PBS), physiological saline, and a FACS wash buffer (buffer containing fetal bovine serum or bovine serum albumin and ethylenediaminetetraacetic acid (EDTA)), but is not limited thereto.

The third centrifugation may be conducted at 4 to 25° C., 4 to 22° C., 4 to 19° C., 4 to 16° C., 4 to 13° C., 4 to 10° C., 4 to 7° C., or 4 to 5° C., for example, at 4° C., but with no limitations thereto.

The third centrifugation may be conducted at 400 to 2000×g, 400 to 1800×g, 400 to 1600×g, 400 to 1400×g, 400 to 1200×g, 400 to 1000×g, or 400 to 800×g, for example, at 441×g, but with no limitations thereto.

In addition, the third centrifugation may be conducted for 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 3 to 60 minutes, 3 to 55 minutes, 3 to 50 minutes, 3 to 45 minutes, 3 to 40 minutes, 3 to 35 minutes, 3 to 30 minutes, 3 to 25 minutes, 3 to 20 minutes, 3 to 15 minutes, or 3 to 10 minutes, for example, 5 minutes, but with no limitations thereto.

In an embodiment of the present disclosure, the third centrifugation may be conducted at 4° C. and 441×g for 5 minutes.

In an embodiment of the present disclosure, the labeled mononuclear cell analyzing step may include the following steps of:

obtaining only single cells;

sorting the cells to give a CD45-positive cell population;

reclassifying the cells to give a HLA-DR-negative cell population;

reclassifying the cells to give a CD11b-positive cell population; and

reclassifying the cells to give a CD14-positive and CCR2-positive cell population.

The analysis may be performed through immunochromatography, immunohistochemical staining, enzyme-linked immunosorbent assay (ELISA), radioimmunoassay (RIA), enzyme immunoassay (EIA), florescence immunoassay (FIA), luminescence immunoassay (LIA), Western blotting, fluorescence activated cell sorter (FACS), or flow cytometry, for example, flow cytometry, but with no limitations thereto.

As used herein, the term “flow cytometry” refers to a technique used to count cells, sort cells, or probe biomarkers. By the technique, cells labeled with a fluorescent or an isotope can be detected.

As used herein, “immunoassay” refers to a biochemical test method for measuring the presence or concentration of a polymer in a solution with the aid of an antibody or immunoglobulin, and includes, for example, radioimmunoassay (RIA), enzyme-linked immunosorbent assay (ELISA), immunofluorescence, and chemiluminescent-linked immunoassay.

The assay method may be used for quantitative analysis of cells by counting cells conjugated with the detecting agent or by measuring concentrations of the cells.

In an embodiment of the present disclosure, the cancer may be breast cancer, prostate cancer, liver cancer, lung cancer, bladder cancer, stomach cancer, uterine cancer, colorectal cancer, colon cancer, blood cancer, ovarian cancer, pancreatic cancer, spleen cancer, testis cancer, thymic carcinoma, brain cancer, esophageal cancer, kidney cancer, cholangiocarcinoma, ovarian cancer, thyroid cancer, or skin cancer, for example, breast cancer or prostate cancer, but is not limited thereto.

### Advantageous Effects of Invention

The present disclosure relates to a composition for diagnosis of cancer bone metastasis, a method for providing information necessary for diagnosis of cancer bone metastasis using same, a method for providing information for monitoring a response to therapy for cancer bone metastasis using same, and a method for screening a therapeutic agent for cancer bone metastasis using same. The composition for diagnosis of cancer bone metastasis according to the present disclosure can more effectively diagnose bone metastasis than whole body bone scan or conventional serum markers for diagnosis of bone metastasis and can thus make early diagnosis of cancer bone metastasis, thereby greatly improving the survival rate of the patients through monitoring of responses to therapy for cancer bone metastasis.

### BRIEF DESCRIPTION OF DRAWINGS

FIG. 1 shows in vivo bioluminescence imaging (hereinafter referred to as “BLI”) results obtained in the procedure of constructing bone micrometastasis mice according to an embodiment of the present disclosure.

FIG. 2 shows images of normal mouse bone tissues in the procedure of constructing bone micrometastasis mice according to an embodiment of the present disclosure.

FIG. 3 shows images of bone tissues in the bone micrometastasis model during the construction of bone micrometastasis mice according to an embodiment of the present disclosure.

FIG. 4 is a plot of overall distribution patterns of mononuclear myeloid-derived suppressor cells in the blood of bone micrometastasis mouse models according to an embodiment of the present disclosure.

FIG. 5 is a plot of distribution patterns of mononuclear myeloid-derived suppressor cells in the blood of bone micrometastasis mouse models according to the present disclosure.

FIG. 6 is a plot of distribution patterns of CCR2+ mononuclear myeloid-derived suppressor cells in the blood of bone micrometastasis mouse models according to the present disclosure.

FIG. 7a is a flow cytometry profile of cancer patients according to an embodiment of the present disclosure.

FIG. 7b is a flow cytometry profile of cancer patients according to an embodiment of the present disclosure.

FIG. 7c is a flow cytometry profile of cancer patients according to an embodiment of the present disclosure.

FIG. 8 is a plot of clinical study results from cancer patient blood samples according to an embodiment of the present disclosure.

### DETAILED DESCRIPTION

Hereinafter, the present disclosure will be described in more detail through examples. The following examples are for illustrative purposes only and it will be apparent to those of ordinary skill in the related art that the scope of this disclosure is not limited by the examples.

### Example 1: Construction of Bone Micrometastasis Model

In order to secure ideal bone metastasis animal models overcoming limits of preexisting bone metastasis mouse models, bone metastasis mouse models were constructed. All experiments relevant to the construction of the mouse models were approved by the Institutional Animal Care and Use Committee in the Korea University College of Medicine and conducted under strict appliance. For constructing the animal models, Balb/c lineage mice were used. The mouse mammary gland cell line 4T1-tdTomato; Luc was subcultured to prepare a sufficient amount of cells to be injected into the mice.

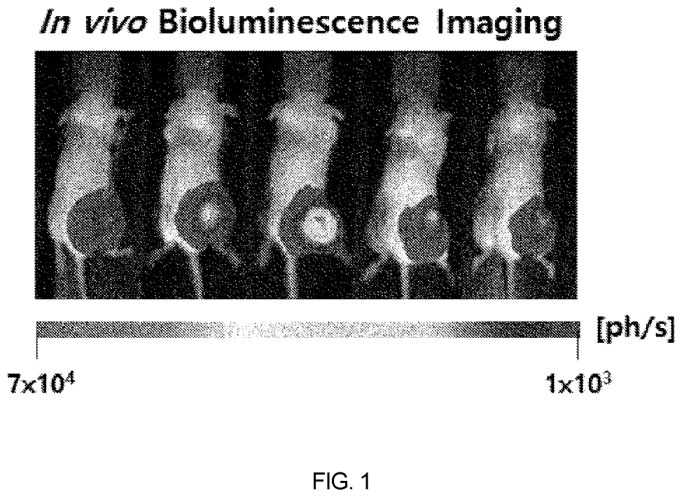

The cancer cells were seeded into one femur or tibia of each mouse by injection through the iliac artery. In brief, after mice were generally anesthetized with 2-3% vaporized isoflurane in conjunction with oxygen, an incision was made into the medial femoral skin to expose the femoral artery. Blood vessels were dissected using an anatomy microscope. The cells were introduced into the blood vessels with the aid of a syringe. After hemostasis and suturing, the mice were laid on heating pad and allowed to sufficiently recover from the surgery. Every week after injection of the cancer cells, the treated femoral and tibial regions were monitored for the formation or growth of metastatic bone tumors by in-vivo bioluminescence imaging according to bioluminescence intensity (ph/s). The results are depicted in FIG. 1.

As can be seen in FIG. 1, the construction of bone micrometastasis mouse models was primarily finished.

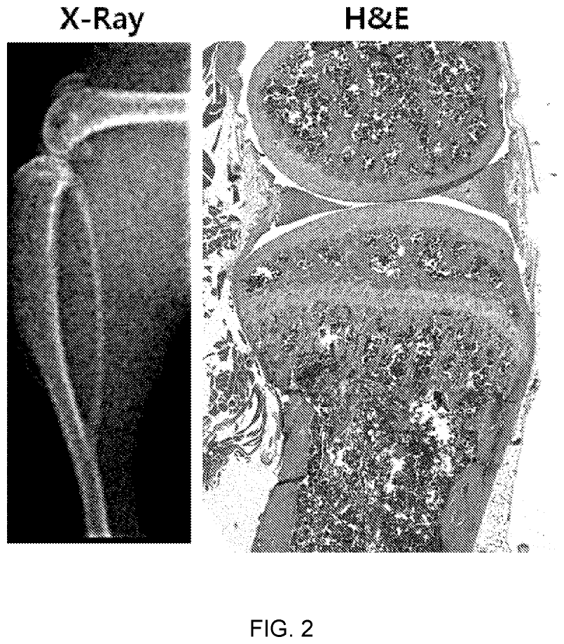

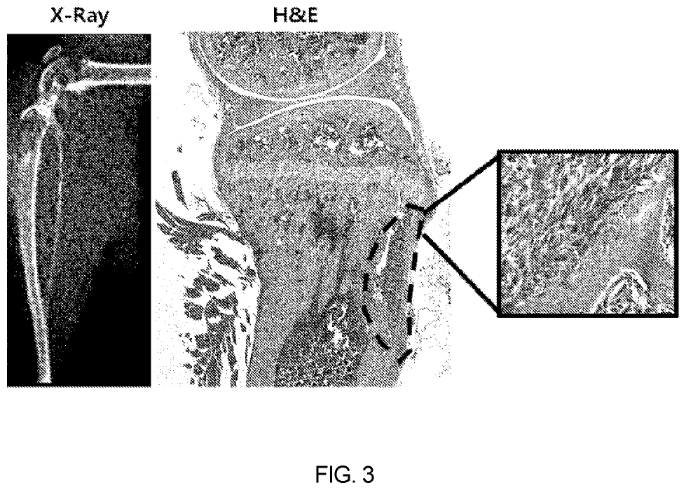

Then, in order to examine whether substantial bone metastasis proceeded, the mice were euthanized and the femur and tibia were separated and histologically analyzed for bone metastasis. Bone metastasis regions were roughly determined in the separated bone tissues by X-ray imaging. The tissues were fixed for 3 days in a 4% paraformaldehyde solution and the muscles attached to the bones were peeled off, followed by decalcification for about two weeks in a 0.5 M EDTA solution. The decalcification was carried out by slowly shaking at 4° C., with change of 0.5 M EDTA with a fresh one every three days. Subsequently, the tissues were embedded into paraffin blocks, sectioned into slices, and mounted onto slides, followed by histological staining with hematoxylin and eosin (H&E). Bioluminescence imaging, X-ray imaging, and histological staining analysis results according to tumor growth are depicted in FIGS. 2 and 3.

As is understood from FIGS. 2 and 3, bone fracture and tumorigenesis were identified in the leg bones of the cancer bone micrometastasis models as measured by X ray and histological staining analysis whereas no tumorigenesis was detected in the bone tissues of normal mice. Therefore, successful construction of cancer bone micrometastasis models was confirmed, as in FIG. 1.

### Example 2: Analysis for Myeloid-Derived Suppressor Cell According to Bone Micrometastasis Growth

For use in analyzing myeloid-derived suppressor cells according to bone metastasis growth by weeks after cancer cell transplantation, blood samples were taken from the bone micrometastasis mouse models constructed in Example 1. A total of 20, 10, and 10 mouse models were prepared for week 1, 2, and 3 after cancer cell transplantation, respectively.

Before blood sampling, the syringe to be used for blood sampling was coated with a heparin solution to prevent blood coagulation. After general anesthesia of the mice, about 1 ml of whole blood was taken from each of the mice via a heart puncture. Immediately after blood sampling, red blood cells were removed by adding the blood to an RBS lysis buffer to lyse red blood cells. The blood was allowed to stand at room temperature for 15 minutes in the RBC lysis buffer. After centrifugation at 441×g for 5 minutes, the supernatant was removed. These procedures were repeated until complete removal of red blood cells. The hemocytes thus clearly separated were stored in a FBS-supplemented buffer in a refrigerator.

For flow cytometry, 10⁶ hemocytes were transferred to each flow cytometer-specific tube, washed, and incubated for 5 minutes with 200 μl of a buffer containing CD16/CD32 antibody to conduct an Fc blocking process. Then, anti-viability dye 780 (1 μl/10⁶ cells), anti-CD45 (0.25 μl/10⁶ cells), anti-CD11b (0.25 μl/10⁶ cells), anti-Ly-6G (0.5 μl/10⁶ cells), anti-Ly-6C (0.5 μl/10⁶ cells), and anti-CCR2 (1 μl/10⁶ cells) antibodies were added to the tube and incubated at room temperature for 30 minutes for immunostaining.

Information on antibodies used:

Viability dye 780 APC-Cy7 (Biogems, #6910-00),

Anti-CD45 PE-Cy7 (Biolegned, #103114),

Anti-CD11 b FITC (BD, #553310),

Anti-Ly-6G Alexa 647 (BD, #12610),

Anti-Ly-6C Alexa 700 (BD, #562137),

Anti-C-C chemokine Receptor 2 (CCR2) APC (Biolegend, #150604).

After completion of the staining, the cells in each tube were washed with about 4 ml of a buffer and then centrifuged at 441×g for 5 minutes to obtain antibody-labeled mononuclear cells. The cells were analyzed by flow cytometry (FACS Canto ± and FACS DIVA software from Beckton-Dickinson).

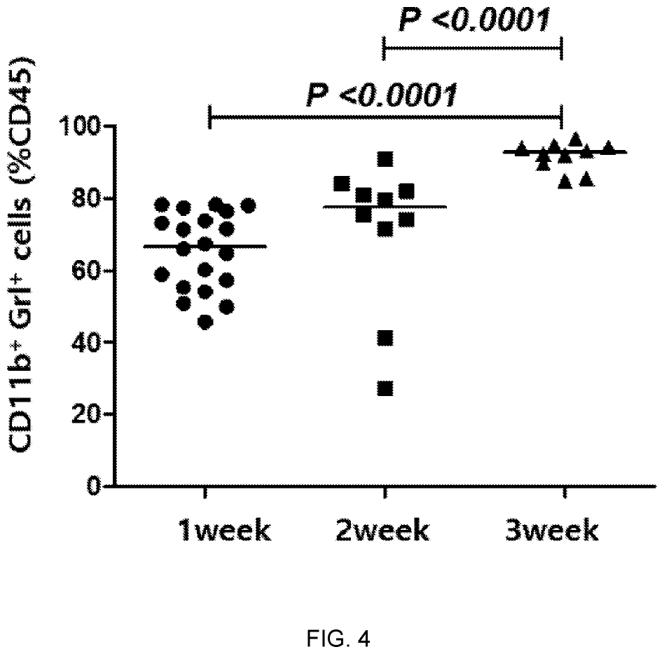

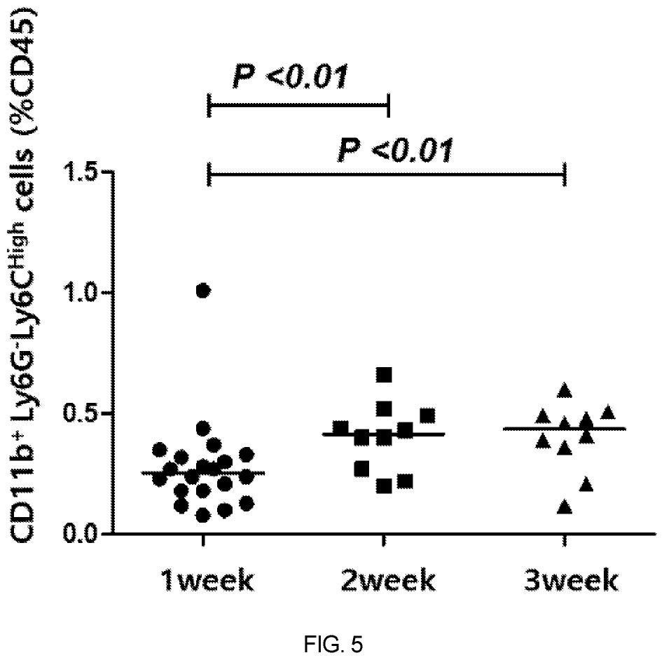

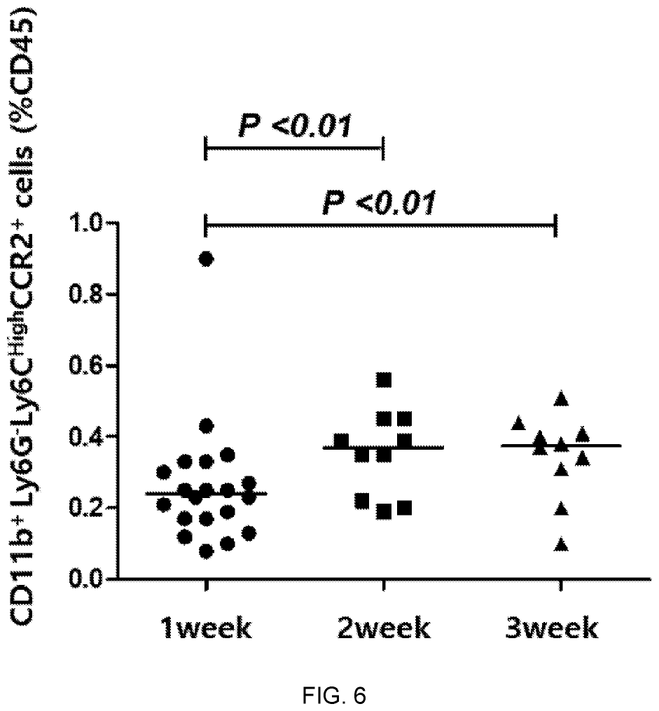

As for analysis, only single cells were obtained first and sorted into cell populations in the order of the antibodies enumerated above. Briefly, among sorted single cell populations, selection was made of viable cells which were then classified in terms of CD45+. Subsequently, reclassification was made of the CD45+ cell population according to CD11b+, Ly-6G-, and Ly-6C+ in that order. Finally, the Ly-6G- and Ly-6C+ cell population was sorted again in terms of Ly-6C+CCR2+ and counted as % values. The results are depicted in FIGS. 4 to 6 and summarized in Tables 1 to 3.

TABLE 1
CD11b (% CD45)

| Week 1 | Week 2 | Week 3 |
|---|---|---|
| 78.19 | 84.05 | 94.65 |
| 78.05 | 82.03 | 94.32 |
| 77.36 | 80.88 | 94.08 |

TABLE 2
Ly-6G−/Ly-6C+ (% CD45)

| Week 1 | Week 2 | Week 3 |
|---|---|---|
| 0.44 | 0.52 | 0.51 |
| 0.37 | 0.49 | 0.49 |
| 0.35 | 0.44 | 0.48 |

TABLE 3
Ly-6C+CCR2+ on CD45 (%)

| Week 1 | Week 2 | Week 3 |
|---|---|---|
| 0.43 | 0.45 | 0.44 |
| 0.35 | 0.45 | 0.41 |
| 0.33 | 0.39 | 0.4 |

As is understood from the data of FIGS. 4 to 6 and Tables 1 to 3, the overall distribution pattern of the myeloid-derived suppressor cells gradually increased with weekly time after cell transplantation. Counts of the mononuclear myeloid-derived suppressor cells and the CCR2+ mononuclear myeloid-derived suppressor cells significantly increased weeks 2 and 3, compared to week 1, but with no significant difference between weeks 2 and 3. This result implies that counts of the mononuclear myeloid-derived suppressor cells already peaked by week 2.

### Example 3. Attainment of Peripheral Blood Mononuclear Cell from Cancer Patient Blood Sample and Establishment of Flow Cytometry

To establish an analysis method for peripheral blood mononuclear cells in human patients, liquid biopsy was conducted to obtained blood samples from cancer patients and to isolate peripheral blood mononuclear cells. For patient blood samples, specimens were attained according to regulations under the approval of the Institutional Review Board (IRB) in the Korea University Anam Hospital Clinical Trial Center.

Briefly, 8 cc of a blood sample was taken from each patient to be diagnosed and transferred to a specific tube coated with heparin for preventing blood coagulation. Then, density-gradient centrifugation using Ficoll was conducted at 4° C. and 635×g for 20 minutes to split layers of plasma, peripheral blood mononuclear cells (hereinafter referred to as “PBMC”), Ficoll, and red blood cells (RBC) in that order. Among the split layers, only the second layer of PBMC was carefully isolated and transferred to a new tube. The PBMC was washed with PBS and then centrifuged at 783×g for 5 minutes to obtain washed PBMC only.

The mononuclear cells were subjected to antibody staining in order to establish flow cytometry for the mononuclear cells. For use in flow cytometry, the peripheral blood mononuclear cells were placed at a density of 2×10⁵ cell/tube in a FACS-specific tube.

Then, the cells were stained by incubation with anti-CD45 (0.5 μl/2×10⁵ cells), anti-CD14 (0.5 μl/2×10⁵ cells), anti-CD15 (5 μl/2×10⁵ cells), anti-HLA-DR (0.5 μl/2×10⁵ cells), anti-CD33 (1.25 μl/2×10⁵ cells), anti-CD11b (2.5 μl/2×10⁵ cells), anti-CCR2 (1 μl/2×10⁵ cells) antibodies at room temperature for 20 minutes.

Information on antibodies used:

Anti-CD45 PerPC-Cy5.5 (BD #564105),

Anti-HLA-DR Alexa700 (BD #560743),

Anti-CD11 b APC (BD #55019),

Anti-CD14 APC-Cy7 (BD #557831),

Anti-CD15 FITC (BD #555401),

Anti-CD33 PE-Cy7 (BD #333946),

Anti-CCR2 PE (Biolegend #357206).

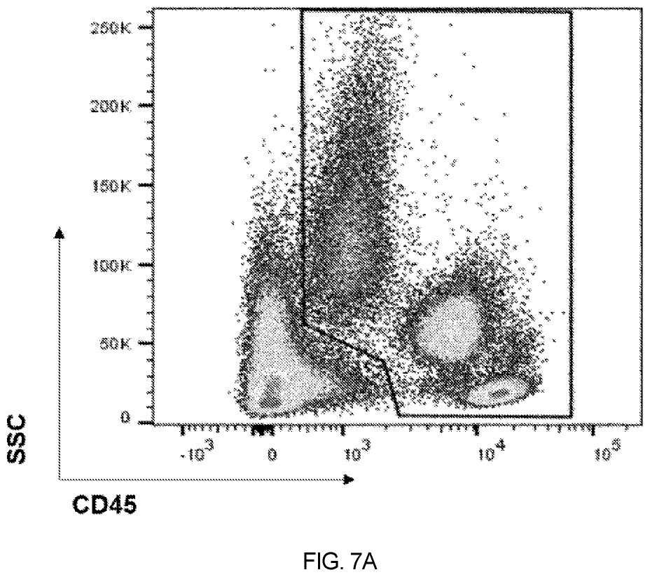

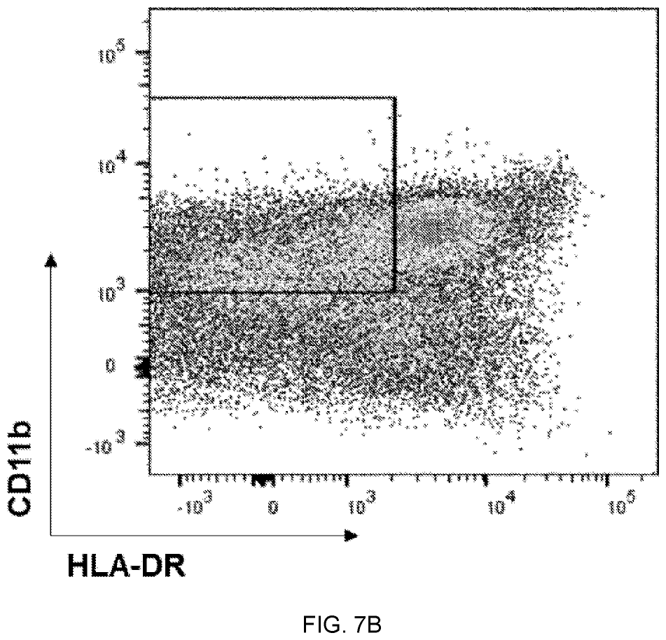

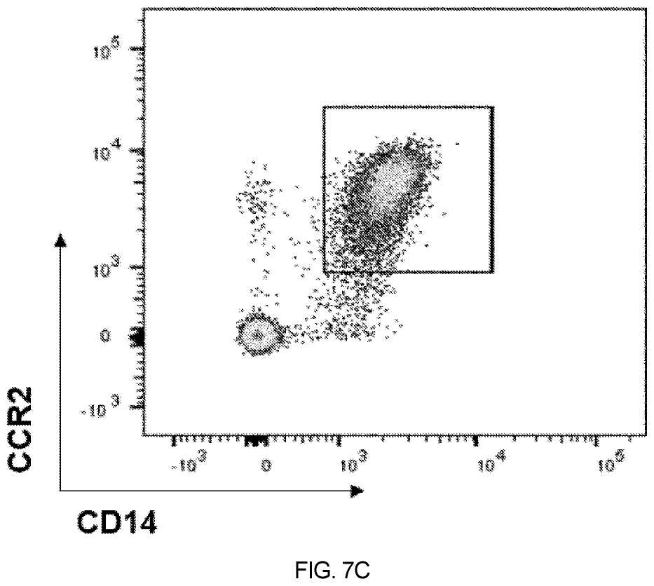

Thereafter, the cells in each tube were washed with about 4 ml of a buffer and then centrifuged at 441×g for 5 minutes to obtain antibody-labeled mononuclear cells. The cells were analyzed by flow cytometry (FACS Canto ± or Fortessa X-20, and FACS DIVA software from Beckton-Dickinson) as in Example 2. For analysis, only single cells were gated first and sorted into cell populations. From corresponding cell populations, selection was made of a CD45+ cell population which was then reclassified on the basis of HLA-DR- and CD11+ only. Subsequently, the HLA-DR- and CD11+ populations were classified on the basis of CD14+ and CCR2+. The resulting cell populations were counted and CCR2+ mononuclear myeloid-derived suppressor cells were analyzed. The analysis results obtained in the cancer patient specimens are depicted in FIG. 7 and summarized in Table 4.

TABLE 4

| Gating | No. of cells | Percentage |
|---|---|---|
| Total | 267,758 | — |
| Single cells | 108,911 | — |
| CD45 | 50,581 | 46.4% (Single) |
| CD11b, HLA-DR- | 17,355 | 34.3% (CD45)) |
| CD14, CCR2 | 7,447 | 14.7% (CD45) |

As can be seen in FIG. 7 and Table 4, a total of 267,758 cells were analyzed. Among them, 108,911 single cells were selected. Then, the single cells were sorted on the basis of CD45+(46.4% relative to the single cells), CD11b+HLA-DR-(34.3% relative to CD45+ cells), and CD14+CCR2+(14.7% relative to CD45+ cells) in that order. For the analytical criteria, a total of 696 breast cancer and prostate cancer patients were analyzed to construct cohorts.

### Example 4. Clinical Study of Mononuclear Myeloid-Derived Suppressor Cells

On the basis of the flow cytometry established in Example 3, mononuclear myeloid-derived suppressor cells present in blood of cancer patients were analyzed, and evaluated for clinically diagnostic value. Counts (%) of individual populations including total counts of myeloid-derived suppressor cells (CD45+CD11b+ cells) and CCR2+ mononuclear myeloid-derived suppressor cells (CD14+CCR2+ cells) were compared.

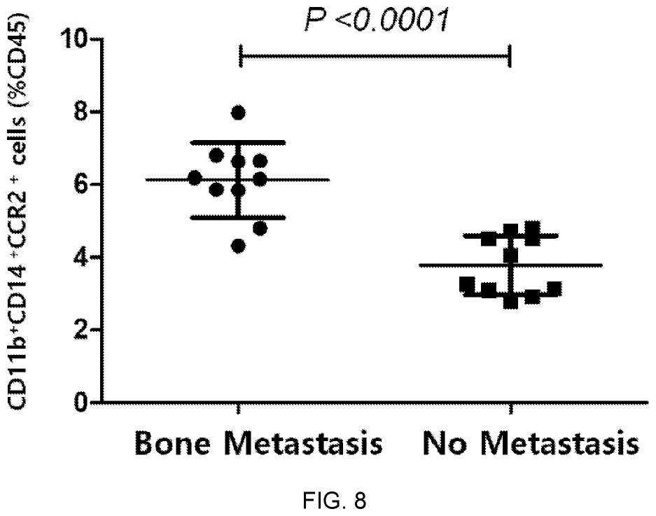

Furthermore, the corresponding cancer patients were examined for metastasis and divided into groups: metastasized to bone, metastasized to other organs, and non-metastasis. Cells in each patient group were counted. Between bone metastasis and other metastasis patients and non-metastasis patients, CCR2+ mononuclear myeloid-derived suppressor cells were analyzed to significantly differ in count, demonstrating the clinical diagnostic value thereof. The results are depicted in FIG. 8 and summarized in Table 5.

TABLE 5
CCR2+ CD14+ M-MDSC

| Bone metastasis | Non-metastasis |
|---|---|
| patient | patient |
| 6.63% | 4.51% |
| 6.18% | 4.04% |
| 6.14% | 3.26% |

As can be seen in FIG. 8 and Table 5, CCR2+ mononuclear myeloid-derived suppressor cells were detected at explicitly higher % in the metastasis group, compared to the non-metastasis group and thus evaluated to have a clinically diagnostic value for bone metastasis lesions.

1. A composition for diagnosis of cancer bone metastasis, comprising:
a CD45 detecting agent, a HLA-DR detecting agent, and a CD11 b detecting agent; and
(a) a CD14 detecting agent and a CCR2 detecting agent, (b) a CD14 detecting agent, (c) a CD15 detecting agent and a CD33 detecting agent, or (d) a CD14 detecting agent, a CCR2 detecting agent, a CD15 detecting agent, and a CD33 detecting agent.

2. The composition of claim 1, wherein the detecting agents are each independently selected from the group consisting of an antibody, an aptamer, DNA, RNA, a protein, and a polypeptide.

3. The composition of claim 1, wherein the detecting agents are each independently labeled with at least one marker selected from the group consisting of a ligand, a bead, a radionuclide, an enzyme, a substrate, a cofactor, an inhibitor, a fluorescer, a fluorescent protein, a chemiluminescent substance, a magnetic particle, a hapten, and a dye.

4. The composition of claim 1, wherein the detecting agents contained in the composition each have a concentration of 5 μl/2×10⁵ cells or more.

5. The composition of claim 1, wherein the cancer is breast cancer, prostate cancer, liver cancer, lung cancer, bladder cancer, stomach cancer, uterine cancer, colorectal cancer, colon cancer, blood cancer, ovarian cancer, pancreatic cancer, spleen cancer, testis cancer, thymic carcinoma, brain cancer, esophageal cancer, kidney cancer, cholangiocarcinoma, ovarian cancer, thyroid cancer, or skin cancer.

6. A method for providing information necessary for diagnosis of cancer bone metastasis, the method comprising:
a labeling step of contacting the composition for diagnosis of cancer bone metastasis of claim 1 with a sample;
an acquiring step of acquiring a labeled mononuclear cell; and
an analyzing step of analyzing the labeled mononuclear cell.

7. The method of claim 6, wherein the sample contains a mononuclear cell isolated from peripheral blood.

8. The method of claim 6, wherein the sample comprises 2×10⁵ or more mononuclear cells.

9. The method of claim 6, wherein the sample is obtained through:
a first centrifugation step for centrifuging a blood sample taken from a patient;
a mononuclear cell layer acquiring step;
a mononuclear cell layer washing step; and
a second centrifugation step.

10. The method of claim 6, wherein the analyzing step comprises the following steps of:
obtaining only single cells;
sorting the cells to give a CD45-positive cell population;
reclassifying the cells to give a HLA-DR-negative cell population;
reclassifying the cells to give a CD11b-positive cell population; and
reclassifying the cells to give a CD14-positive and CCR2-positive cell population.

11. A method for providing information for monitoring a response to therapy for cancer bone metastasis, the method comprising:
a labeling step of contacting a composition for diagnosis of cancer bone metastasis with a sample;
an acquiring step of acquiring a labeled mononuclear cell; and
an analyzing step of analyzing the labeled mononuclear cell.

12. (canceled)
